(19) United States
(12) Patent Application Publication
(10) Pub. No.: US 2025/0214959 A1
(43) Pub. Date: Jul. 3, 2025

(54) THIN FILM SHIELDING AGENT, METHOD OF FORMING THIN FILM USING THIN FILM SHIELDING AGENT, SEMICONDUCTOR SUBSTRATE INCLUDING THIN FILM, AND SEMICONDUCTOR DEVICE INCLUDING SEMICONDUCTOR SUBSTRATE

(71) Applicant: SOULBRAIN CO., LTD., Gyeonggi-do (KR)

(72) Inventors: Seung Hyun LEE, Gyeonggi-do (KR); Jae Sun JUNG, Gyeonggi-do (KR); Chang Bong YEON, Gyeonggi-do (KR); Deok Hyun KIM, Gyeonggi-do (KR)

(21) Appl. No.: 18/848,682

(22) PCT Filed: Mar. 17, 2023
(86) PCT No.: PCT/KR2023/003579

§ 371 Date: Sep. 19, 2024

(30) Foreign Application Priority Data
Apr. 5, 2022 (KR) 10-2022-0042356
Oct. 25, 2022 (KR) 10-2022-0138092

(51) Int. Cl. C07D 327/10; C23C 16/04; C23C 16/455; H01L 21/02

(52) U.S. Cl. CPC C07D 327/10; C23C 16/04; C23C 16/45527; H01L 21/0228

(57) ABSTRACT

The present invention relates to a thin film shielding agent, a thin film-forming composition including the thin film shielding agent, a method of forming a thin film using the thin film shielding agent, a semiconductor substrate including the thin film, and a semiconductor device including the semiconductor substrate. According to the present invention, by applying the thin film shielding agent, the reaction speed may be improved, and the thin film growth rate may be appropriately reduced. Accordingly, even when a thin film is formed on a substrate having a complex structure under high-temperature conditions, step coverage and the thickness uniformity of the thin film may be greatly improved, and a seamless thin film may be formed. In addition, by reducing impurities, film quality may be improved.

### TECHNICAL FIELD

The present invention relates to a thin film shielding agent, a method of forming a thin film using the thin film shielding agent, a semiconductor substrate including the thin film, and a semiconductor device including the semiconductor substrate. More particularly, the present invention relates to a thin film shielding agent being capable of providing a seamless thin film by significantly improving step coverage and the thickness uniformity of a thin film even when a thin film is formed under high-temperature conditions on a substrate having a complex structure by providing strong and stable physical or chemical adsorption to the surface of a deposition layer on the substrate; and being capable of significantly reducing impurities and improving film quality, a method of forming a thin film using the thin film shielding agent, and a semiconductor substrate including the thin film.

### BACKGROUND ART

As the integration of memory and non-memory semiconductor devices increases, the microstructure of a substrate is becoming increasingly complex.

For example, the ratio of the width to depth of the microstructure (hereinafter referred to as the ‘aspect ratio’) is increasing to 20:1 or more, and even to 100:1 or more. As the aspect ratio increases, it becomes difficult to form a deposition layer with a uniform thickness along the plane of the complex microstructure.

Accordingly, step coverage, which defines the thickness ratio of deposition layers formed at the top and bottom in the depth direction of the microstructure, remains at the level of 90%. Accordingly, the expression of the electrical characteristics of a device becomes increasingly difficult. Since a step coverage of 100% means that deposition layers formed on the upper and lower parts of the microstructure have the same thickness, it is necessary to develop a technology that can achieve step coverage close to 100%.

In the case of a word line of NAND made of metal, when a very narrow gap (groove) is filled, when the contents of the metal accumulated at the top and bottom of the gap are different, and the metal is dominantly accumulated at the top, it can cause a seam defect in which an empty space is created inside the gap.

To make step coverage, which is a variable that determines whether the thicknesses of atomic layers deposited on the upper and lower parts of a pattern are the same, close to 100%, it is necessary to induce a difference in the adsorption density of a shielding agent for a precursor compound so that the precursor compound occupies more adsorption sites at the top of the pattern and less sites toward the bottom.

In addition, to implement excellent film quality, the problem of difficulty in adsorbing a shielding agent onto a substrate as deposition temperature increases must be overcome.

As a specific example, according to Korean Patent Application Publication No. 2021-0059332, which uses ether as a shielding agent material, instead of providing a thin film shielding effect, there is a problem of remaining oxygen or carbon impurities in a deposition layer due to hydrogen, which is a typical reactant used in forming a metal layer, or ammonia, which is a typical reactant used in forming a nitride metal layer.

Therefore, there is a need to develop a method of effectively forming a thin film having a complex structure even in high temperatures, reducing impurities, and forming a seamless thin film by greatly improving step coverage and the thickness uniformity of a thin film, and a semiconductor substrate fabricated using the same.

### DISCLOSURE

### Technical Problem

Therefore, the present invention has been made in view of the above problems, and it is one object of the present invention to provide a thin film shielding agent being capable of forming a seamless thin film by significantly improving step coverage and the thickness uniformity of a thin film even when a thin film is formed under high-temperature conditions on a substrate having a complex structure by providing strong and stable physical or chemical adsorption to the surface of a deposition layer on the substrate regardless of deposition temperature; and being capable of reducing impurities and improving film quality.

It is another object of the present invention to provide a thin film formation method that improves the density, electrical properties, and dielectric properties of a thin film by improving the crystallinity and oxidation fraction of the thin film and a semiconductor substrate including the thin film.

The above and other objects can be accomplished by the present invention described below.

### Technical Solution

In accordance with one aspect of the present invention, provided is a thin film shielding agent for depositing a metal oxide film or non-metal oxide film on a substrate, wherein the thin film shielding agent includes one or more elements having electronegativity between electronegativity of a metal or non-metal constituting the oxide film and electronegativity of oxygen and shields the deposition.

The metal or non-metal may include one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

The thin film shielding agent may be a compound including four or more elements having an electronegativity of 2.1 to 3.1.

The thin film shielding agent may be a compound having a structure represented by Chemical Formula 1 below.

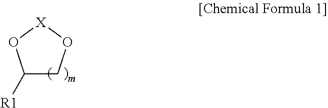

In Chemical Formula 1, R1 is H, OH, CH₃, OCH₃, OCH₂CH₃, OCH₂CH₂CH₃, an alkyl group having 1 to 5 carbon atoms, an alkene group having 1 to 5 carbon atoms, or an alkane group having 1 to 5 carbon atoms; X is

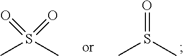

and m is an integer from 0 to 4.

The thin film shielding agent may have a refractive index of 1.4 or more, 1.5 or less, 1.41 to 1.48, or 1.41 to 1.47.

The thin film shielding agent may include one or more selected from compounds represented by Chemical Formulas 1-1 to 1-9 below.

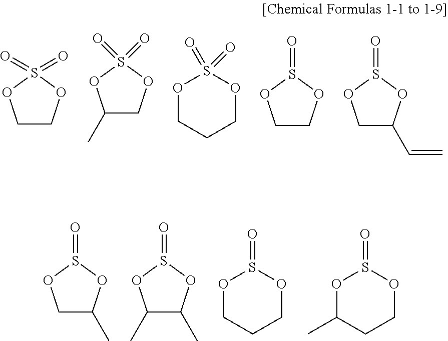

In accordance with another aspect of the present invention, provided is a thin film-forming composition including:
a precursor compound constituting a thin film deposition layer; and a thin film shielding agent,
wherein the thin film shielding agent is the above-described thin film shielding agent; and the precursor compound is a compound represented by Chemical Formula 2 below.

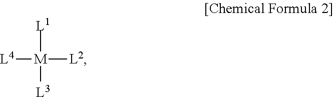

wherein M includes one or more selected from Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd; and L1, L2, L3, and L4 are —H, —X, —R, —OR, —NR, or Cp (cyclopentadiene) and are the same or different, wherein —X is F, Cl, Br, or I; —R is C1-C10 alkyl, C1-C10 alkene, or C1-C10 alkane and is linear or cyclic; and L1, L2, L3, and L4 are formed from 2 to 6 depending on an oxidation number of a central metal (M).

For example, when the central metal is divalent, L1 and L2 may be attached to the central metal as ligands. When the central metal is hexavalent, L1, L2, L3, L4, L5, and L6 may be attached to the central metal. Ligands corresponding to L1 to L6 may be the same or different.

The thin film shielding agent may provide a shielded area for an oxide film, a nitride film, a metal film, or a selective thin film thereof, and the shielded area may be formed on the entire substrate or a portion of the substrate on which the oxide film, the nitride film, the metal film, or the selective thin film thereof is formed.

Based on 100% of a total area of the substrate, the shielded area may occupy 10 to 95% of the entire substrate or a portion of the substrate, and an unshielded area may occupy the remainder.

The thin film may improve step coverage in a process of forming a laminated film of one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

The thin film may be used as a diffusion barrier film, an etching stop film, an electrode film, a dielectric film, a gate insulating film, a block oxide film, or a charge trap.

In accordance with still another aspect of the present invention, provided is a method of forming a thin film, the method including injecting one or more thin film shielding agents selected from compounds having a structure represented by Chemical Formula 1 below and a precursor compound into a chamber to form a deposition layer on a substrate loaded into the chamber.

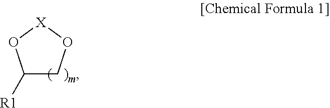

wherein R1 is H, OH, CH₃, OCH₃, OCH₂CH₃, OCH₂CH₂CH₃, an alkyl group having 1 to 5 carbon atoms, an alkene group having 1 to 5 carbon atoms, or an alkane group having 1 to 5 carbon atoms; X is

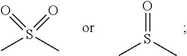

and m is an integer from 0 to 4.

The chamber may be an ALD chamber, a CVD chamber, a PEALD chamber, or a PECVD chamber.

The precursor compound and the thin film shielding agent may be independently transported into the chamber by a VFC, DLI, or LDS method.

The thin film may be a silicon nitride film, a silicon oxide film, a titanium nitride film, a titanium oxide film, a tungsten nitride film, a molybdenum nitride film, a hafnium oxide film, a zirconium oxide film, a tungsten oxide film, or an aluminum oxide film.

The thin film shielding agent may have a deposition rate reduction rate of 20% or more as calculated by Equation 1 below.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a thin film shielding agent. DR_{f}(final deposition rate) is the deposition rate of the thin film formed by adding an oxide film thin film shielding agent during the above process. Here, the deposition rate (DR) is a value measured at room temperature and pressure using an ellipsometer for a thin film with a thickness of 3 to 30 nm, and is expressed in a unit of Å/cycle.

The thin film shielding agent may provide a substitution area for an oxide film, a nitride film, a metal film, or a selective thin film thereof.

The substitution area may be formed on the entire or part of a substrate on which the oxide film, the nitride film, the metal film, or the selective thin film thereof is formed.

Based on 100% of a total area of the substrate, the ligand adsorbed area may occupy 10 to 95% of the entire substrate or a portion of the substrate, and a ligand non-adsorbed area may occupy a remainder.

Based on 100% of a total area of the substrate, a first ligand adsorbed area may occupy 10 to 95% of the entire substrate or a portion of the substrate, a second ligand adsorbed area may occupy 10 to 95% of a remaining area, and the ligand non-adsorbed area may occupy a remainder.

The thin film may be a laminated film of one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

The thin film may be used as a diffusion barrier film, an etching stop film, an electrode film, a dielectric film, a gate insulating film, a block oxide film, or a charge trap, and may improve step coverage during a formation process thereof.

A precursor compound used in the method of forming a thin film may be a compound represented by Chemical Formula 2 below.

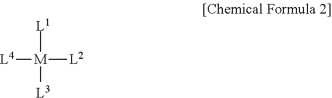

In Chemical Formula 2, M includes one or more selected from Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd; and L1, L2, L3, and L4 are —H, —X, —R, -Cp, —OR, —NR, or Cp (cyclopentadiene) and are the same or different. Here, —X is F, Cl, Br, or I; —R is C1-C10 alkyl, C1-C10 alkene, or C1-C10 alkane and is linear or cyclic; and L1, L2, L3, and L4 are formed from 2 to 6 depending on an oxidation number of a central metal (M).

In Chemical Formula 2, L1, L2, L3, and L4 are —H, -Cp, or —R, and are the same or different. Here, —R is C1-C10 alkyl, C1-C10 alkene, or C1-C10 alkane, and is linear or cyclic.

In Chemical Formula 2, L1, L2, L3, and L4 are —H, -Cp, —OR, —NR, or Cp (cyclopentadiene), and are the same or different. Here, —R is H, C1-C10 alkyl, C1-C10 alkene, C1-C10 alkane, iPr, or TBu.

In Chemical Formula 2, L1, L2, L3, and L4 are —H, -Cp, or —X, and are the same or different. Here, —X is F, Cl, Br, or I.

In accordance with still another aspect of the present invention, provided is a method of forming a thin film, the method including:
i) vaporizing the above-described thin film shielding agent to shield a surface of a substrate loaded in a chamber;
ii) performing 1st purging inside the chamber with a purge gas;
iii) vaporizing a precursor compound and adsorbing the precursor compound onto an area outside the shielded area;
iv) performing 2nd purging inside the chamber with a purge gas;
v) supplying a reaction gas inside the chamber; and
vi) performing 3rd purging inside the chamber with a purge gas.

The precursor compound may be a molecule composed of one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd, and may be a precursor having a vapor pressure of greater than 0.01 mTorr and 100 Torr or less at 25° C.

The chamber may be an ALD chamber, a CVD chamber, a PEALD chamber, or a PECVD chamber.

The thin film shielding agent or the precursor compound may be vaporized, injected, and then subjected to plasma post-treatment.

In steps i) and iv), an amount of the purge gas injected into the chamber may be 10 to 100,000 times a volume of thin film shielding agent injected.

The reaction gas may be an oxidizing agent, a nitriding agent, or a reducing agent, and the reactant gas, the thin film shielding agent, and the precursor compound may be transported into the chamber by a VFC, DLI, or LDS method.

The thin film may be a silicon nitride film, a silicon oxide film, a titanium nitride film, a titanium oxide film, a tungsten nitride film, a molybdenum nitride film, a hafnium oxide film, a zirconium oxide film, a tungsten oxide film, or an aluminum oxide film.

The substrate loaded into the chamber may be heated to 100 to 800° C., and the ratio of the amount (mg/cycle) of the thin film shielding agent and the precursor compound fed into the chamber may be 1:1 to 1:20.

In accordance with still another aspect of the present invention, provided is a semiconductor substrate including the thin film formed by the method of forming a thin film described above.

The thin film may have a multilayer structure of two or more layers.

In accordance with yet another aspect of the present invention, provided is a semiconductor device including the above-described semiconductor substrate.

The semiconductor substrate may be low resistive metal gate interconnects, high aspect ratio 3D metal-insulator-metal capacitor, DRAM trench capacitor, 3D Gate-All-Around (GAA), or 3D NAND flash memory.

### Advantageous Effects

According to the present invention, the present invention has an effect of providing a thin film shielding agent that improves step coverage even when forming a thin film on a substrate with a complex structure under high-temperature conditions by effectively shielding adsorption on the surface of the substrate to improve reaction speed and reduce a thin film growth rate appropriately.

In addition, by effectively reducing process by-products during thin film formation, corrosion and deterioration can be prevented, and the film quality can be improved to improve the crystallinity of a thin film, thereby improving the electrical properties of the thin film.

In addition, the step coverage and density of a thin film can be improved. Furthermore, the present invention has an effect of providing a method of forming a thin film using the thin film shielding agent and a semiconductor substrate including the thin film.

### DESCRIPTION OF DRAWINGS

FIG. 1 is a schematic diagram showing a deposition process sequence according to the present invention, focusing on one cycle.

FIG. 2 includes TEM images showing deposition thicknesses at the upper portion (100 nm below the top) and lower portion (100 nm above the bottom) of the cross-section of an oxide film deposited without using a thin film shielding agent according to Comparative Example 1 and deposition thicknesses at the upper portion (100 nm below the top) and lower portion (100 nm above the bottom) of the cross-section of an oxide film deposited using a thin film shielding agent.

FIG. 3 includes TEM images showing deposition thicknesses at the upper portion (100 nm below the top) and lower portion (100 nm above the bottom) of the cross-section of an oxide film deposited using a thin film shielding agent according to Example 1 and deposition thicknesses at the upper portion (100 nm below the top) and lower portion (100 nm above the bottom) of the cross-section of an oxide film deposited using a thin film shielding agent.

FIG. 4 includes TEM images showing deposition thicknesses at the upper portion (100 nm below the top) and lower portion (100 nm above the bottom) of the cross-section of an oxide film deposited using a thin film shielding agent according to Example 2 and deposition thicknesses at the upper portion (100 nm below the top) and lower portion (100 nm above the bottom) of the cross-section of an oxide film deposited using a thin film shielding agent.

### BEST MODE

Hereinafter, a thin film shielding agent of the present invention, a method of forming a thin film using the thin film shielding agent, and a semiconductor substrate including the thin film will be described in detail.

In the present disclosure, unless otherwise specified, the term “shielding” means reducing, inhibiting, or blocking adsorption of precursor compounds for forming a thin film onto a substrate, and also reducing, inhibiting, or blocking adsorption of process by-products onto the substrate.

In the present disclosure, unless otherwise specified, the terms “physical adsorption” and “chemical adsorption” can be divided into adsorption that can be removed by purging or adsorption that remains after purging. The former is referred to as physical adsorption, and the latter as chemical adsorption.

The present inventors confirmed that, when using a thin film shielding agent capable of effectively shielding the adsorption of a precursor compound supplied to form a thin film on the surface of a substrate loaded inside a chamber, the thin film shielding agent improved reaction speed by a strong and stable physical or chemical adsorption mechanism on the deposition layer surface of the substrate. In addition, even when high-temperature conditions were applied to the substrate with a complex structure, film quality was improved, ensuring the uniformity of the thin film and greatly improving step coverage. In particular, it was possible to deposit in a thin thickness, and the remaining O, Si, metal, metal oxide as process by-products, and even the carbon residue that was difficult to reduce in the past were improved. Based on these results, the present inventors conducted further studies to complete the present invention.

For example, the thin film may be provided with one or more precursors selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd, and may provide an oxide film, a nitride film, or a metal film. In this case, the effects desired in the present invention may be sufficiently achieved.

As a specific example, the thin film may have a film composition of a silicon nitride film, a silicon oxide film, a titanium nitride film, a titanium oxide film, a tungsten nitride film, a molybdenum nitride film, a hafnium oxide film, a zirconium oxide film, a tungsten oxide film, or an aluminum oxide film.

The thin film may contain the aforementioned film composition alone or in a selective area, but is not limited thereto, and also includes SiH and SiOH.

In addition to a commonly used diffusion barrier film, the thin film may be used in semiconductor devices as an etching stop film, an electrode film, a dielectric film, a gate insulating film, a block oxide film, or a charge trap.

In the present invention, the precursor compound used in the formation of the thin film may be a molecule having Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, or Nd as a central metal atom (M) and one or more ligands described later. For a precursor with a vapor pressure of 1 mTorr to 100 Torr at 25° C., the effect of substitution with a thin film shielding agent may be maximized.

For example, a compound represented by Chemical Formula 2 below may be used as the precursor compound.

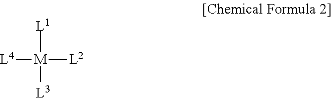

In Chemical Formula 2, M includes one or more selected from Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd; and L1, L2, L3, and L4 are —H, —X, —R, -Cp (cyclopentadienyl), —OR, —NR, or Cp (cyclopentadiene) and are the same or different. Here, —X is F, Cl, Br, or I; —R is C1-C10 alkyl, C1-C10 alkene, or C1-C10 alkane and is linear or cyclic; and L1, L2, L3, and L4 are formed from 2 to 6 depending on an oxidation number of a central metal (M).

For example, when the central metal is divalent, L1 and L2 may be attached to the central metal as ligands. When the central metal is hexavalent, L1, L2, L3, L4, L5, and L6 may be attached to the central metal. The ligands corresponding to L1 to L6 may be the same or different.

In Chemical Formula 2, M is hafnium (Hf), silicon (Si) zirconium (Zr), or aluminum (Al), preferably hafnium (Hf) or silicon (Si). In this case, the effect of reducing process by-products and the effect of improving thin film density may be increased, and step coverage and the electrical properties, insulating properties, and dielectric properties of the thin film may be excellent.

L1, L2, L3, and L4 may be —H, -Cp, or —R and may be the same or different. Here, —R may be C1-C10 alkyl, C1-C10 alkene, or C1-C10 alkane and may have a linear or cyclic structure.

In addition, L1, L2, L3, and L4 may be —H, -Cp, —OR, —NR, or Cp (cyclopentadiene) and may be the same or different. Here, —R may be H, C1-C10 alkyl, C1-C10 alkene, C1-C10 alkane, iPr, or tBu.

In addition, in Chemical Formula 1, L1, L2, L3, and L4 may be —H, -Cp, or —X and may be the same or different.

Specifically, examples of the hafnium precursor compound may include tris(dimethylamido)cyclopentadienyl hafnium of CpHf(NMe₂)₃ and (methyl-3-cyclopentadienylpropylamino)bis(dimethylamino)hafnium of Cp (CH₂)₃NM₃Hf(NMe₂)₂.

In addition, examples of the silicon precursor compound may include one or more selected from SiH₄, SiHCl₃, SiH₂Cl₂, SiCl₄, Si₂Cl₆Si₃Cl₈, Si₄Cl₁₀, SiH₂[NH(C₄H₉)]₂, Si₂(NHC₂H₅)₄, Si₃NH₄(CH₃)₃, SiH₃[N(CH₃)₂], SiH₂[N(CH₃)₂]₂, SiH[N(CH₃)₂]3, and Si[N(CH₃)₂]4.

In addition, examples of the aluminum precursor compound may include trimethyl aluminum (TMA), tris(dimethylamido)aluminum (TDMAA), and aluminum chloride (AlCl₃).

The thin film shielding agent may be used to deposit a metal oxide film or a non-metal oxide film on a substrate.

The thin film shielding agent includes one or more elements having electronegativity between the electronegativity of a metal or non-metal constituting the oxide film and the electronegativity of oxygen, and is used to deposit a metal oxide film or non-metal oxide film.

For example, the metal or non-metal may shield the surface of a thin film formed using one or more precursor compounds selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

When the thin film shielding agent is a compound including four or more elements having an electronegativity of 2.1 to 3.1, the effect of reducing process by-products and the effect of improving thin film density may be increased, and step coverage and the electrical properties of the thin film may be excellent.

Preferably, the thin film shielding agent may be a compound containing sulfur, phosphorus, or nitrogen. In this case, by suppressing side reactions during thin film formation and controlling the thin film growth rate, process by-products within the thin film may be reduced, thereby reducing corrosion and deterioration. In addition, film quality including thin film crystallinity may be improved. In addition, even when a thin film is formed on a substrate having a complex structure, step coverage and the thickness uniformity of a thin film may be greatly improved.

The thin film shielding agent, preferably a compound including four or more elements having an electronegativity of 2.1 to 3.1 may have a deposition rate reduction rate of 20% or more, as a specific example, 35% or more, as calculated by Equation 1 below. In this case, by forming a deposition layer with a uniform thickness due to the difference in the adsorption distribution of the shielding agent having the aforementioned structure as a shielded area that does not remain in a thin film, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a thin film shielding agent. DR_{f}(final deposition rate) is the deposition rate of the thin film formed by adding an oxide film thin film shielding agent during the above process. Here, the deposition rate (DR) is a value measured at room temperature and pressure using an ellipsometer for a thin film with a thickness of 3 to 30 nm, and is expressed in a unit of Å/cycle.

In Equation 1, when the thin film shielding agent is used and when the thin film shielding agent is not used, for each case, the thin film growth rate per cycle means the thin film deposition thickness (Å/cycle) per cycle, i.e., the deposition rate. For example, the deposition rate may be obtained as an average deposition rate calculated by measuring the final thickness of a thin film with a thickness of 3 to 30 nm under room temperature and pressure conditions using an ellipsometer, and then dividing the final thickness by the total number of cycles.

In Equation 1, “when the thin film shielding agent is not used” means that a thin film is manufactured by adsorbing only a precursor compound on a substrate in a thin film deposition process. As a specific example, in the thin film forming method, the above case refers to a case where a thin film is formed by omitting a step of adsorbing a thin film shielding agent and a step of purging an unadsorbed thin film shielding agent.

The thin film shielding agent has two or more types of nitrogen (N), oxygen (O), phosphorus (P), or sulfur (S), and includes a linear or cyclic saturated or unsaturated hydrocarbon having 3 to 15 carbon atoms. In this case, by forming a shielded area that does not remain in a thin film during thin film formation, a relatively coarse thin film may be formed and side reactions may be suppressed. In addition, by controlling the thin film growth rate, process by-products within the thin film may be reduced, thereby reducing corrosion and deterioration. In addition, the crystallinity of the thin film may be improved, and a stoichiometric oxidation state may be reached when a metal oxide film is formed. In addition, even when a thin film is formed on a substrate having a complex structure, step coverage and the thickness uniformity of a thin film may be greatly improved.

The thin film shielding agent includes a central atom connected by a double bond to oxygen, preferably a compound that contains oxygen (O) or phenyl group at one or both terminals of sulfur (S), phosphorus (P), or nitrogen (N), or has a ring structure in which both terminals are connected. Accordingly, the effect of reducing process by-products and the effect of improving thin film density may be increased, and step coverage and the electrical properties of the thin film may be excellent.

As a specific example, the thin film shielding agent may include one or more selected from compounds having a structure represented by Chemical Formula 1 below. In this case, by forming a shielded area that does not remain in a thin film during thin film formation, a relatively coarse thin film may be formed and side reactions may be suppressed. In addition, by controlling the thin film growth rate, process by-products within the thin film may be reduced, thereby reducing corrosion and deterioration. In addition, the crystallinity of the thin film may be improved. In addition, even when a thin film is formed on a substrate having a complex structure, step coverage and the thickness uniformity of a thin film may be greatly improved, and a seamless thin film may be formed.

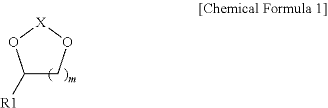

In Chemical Formula 1, R1 is H, OH, CH₃, OCH₃, OCH₂CH₃, OCH₂CH₂CH₃, an alkyl group having 1 to 5 carbon atoms, an alkene group having 1 to 5 carbon atoms, or an alkane group having 1 to 5 carbon atoms; X is

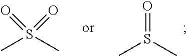

and m is an integer from 0 to 4.

In Chemical Formula 1, R1 is H or CH₃. In this case, the effect of reducing process by-products and the effect of improving thin film density may be increased, and step coverage and the electrical properties, insulating properties, and dielectric properties of the thin film may be excellent.
m is an integer from 0 to 2, preferably 0 or 1.

For example, the thin film shielding agent may be a compound having a refractive index of 1.4 or more, 1.5 or less, 1.41 to 1.48, or 1.41 to 1.47.

In this case, by appropriately shielding the adsorption of precursor compound on the substrate by a strong and stable physical or chemical adsorption mechanism on the deposition layer surface of the substrate with a thin film shielding agent having the structure described above, reaction speed may be improved. In addition, even when forming a thin film under high-temperature conditions on a substrate with a complex structure, step coverage and the thickness uniformity of a thin film may be greatly improved. In addition to a thin film precursor, the surface of the substrate may be effectively protected by preventing the adsorption of process by-products, and process by-products may be effectively removed.

For example, when the shielding agent is a compound represented by Chemical Formula 1, the shielding agent may be a compound having a refractive index of 1.4 or more, 1.5 or less, 1.41 to 1.48, or 1.41 to 1.47.

In this case, by forming, as a shielded area that does not remain in the thin film, a deposition layer of uniform thickness due to the difference in adsorption distribution of the shielding agent having the aforementioned structure on the substrate, the deposition rate of the thin film may be reduced, and the thin film growth rate may be appropriately reduced. Thus, even when forming a thin film under high-temperature conditions on a substrate with a complex structure, step coverage and the thickness uniformity of a thin film may be greatly improved, and a seamless thin film may be formed. In addition to a thin film precursor, the surface of the substrate may be effectively protected by preventing the adsorption of process by-products, and process by-products may be effectively removed.

In particular, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

The thin film shielding agent may include one or more selected from compounds represented by Chemical Formulas 1-1 to 1-9 below. In this case, by providing a thin film-shielded area, the growth rate of a thin film may be effectively controlled, and process by-products may be effectively removed. In addition, step coverage and film quality may be greatly improved.

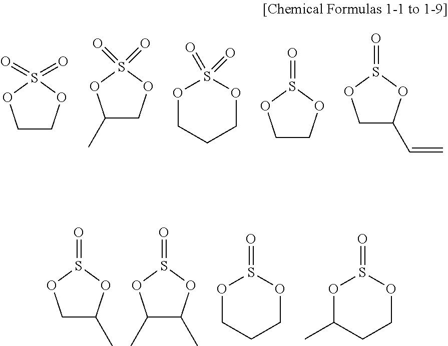

The thin film-forming composition including the thin film shielding agent may include a precursor compound constituting a thin film deposition layer, and the thin film shielding agent.

The precursor compound may be a compound represented by Chemical Formula 2 below.

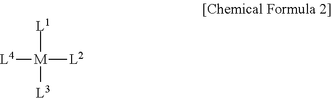

In Chemical Formula 2, M includes one or more selected from Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd; and L1, L2, L3, and L4 are —H, —X, —R, -Cp, —OR, —NR, or Cp (cyclopentadiene) and are the same or different. Here, —X is F, Cl, Br, or I; —R is C1-C10 alkyl, C1-C10 alkene, or C1-C10 alkane and is linear or cyclic; and L1, L2, L3, and L4 are formed from 2 to 6 depending on an oxidation number of a central metal (M).

For example, when the central metal is divalent, L1 and L2 may be attached to the central metal as ligands. When the central metal is hexavalent, L1, L2, L3, L4, L5, and L6 may be attached to the central metal. The ligands corresponding to L1 to L6 may be the same or different.

The thin film shielding agent may provide a shielded area for an oxide film, a nitride film, a metal film, or a selective thin film thereof, and the shielded area may be formed on the entire or part of a substrate on which the oxide film, the nitride film, the metal film, or the selective thin film thereof is formed.

The shielded area for thin films does not remain on the thin film.

At this time, unless otherwise specified, non-residue refers to a case where the content of C element is less than 0.1 atom %, the content of Si element is less than 0.1 atom %, the content of N element is less than 0.1 atom %, and the content of halogen element is less than 0.1 atom % when analyzed by XPS. More preferably, in the secondary-ion mass spectrometry (SIMS) measurement method or X-ray photoelectron spectroscopy (XPS) measurement method, in which measurements are performed in the depth direction of a substrate, considering the increase/decrease rates of C, N, Si, and halogen impurities before and after using an activator under the same deposition conditions, it is desirable that the increase/decrease rate of the signal sensitivity (intensity) of each element type does not exceed 5%.

As a specific example, based on 100% of a total area of the substrate, the shielded area may occupy 10 to 95% of the entire substrate or a portion of the substrate, and an unshielded area may occupy the remainder.

For example, the thin film may include a halogen compound in an amount of 100 ppm or less. For reference, when excess halogen remains, it is not desirable because impurities with a high boiling point, such as NH₄Cl, may be generated and may remain in the thin film when using a nitriding agent under the experimental conditions of 200 to 300° C. described later.

The thin film may be used as an etching stop film, an electrode film, a dielectric film, a gate insulating film, a block oxide film, or a charge trap, and may improve step coverage during the formation process thereof. Thus, the thin film may be used in a semiconductor device.

The thin film shielding agent may be preferably a compound having a purity of 99.9% or more, 99.95% or more, or 99.99% or more. For reference, when a compound having a purity of less than 99% is used, impurities may remain in a thin film or cause side reactions with precursors or reactants. Accordingly, it is desirable to use a material having a purity of 99% or more.

The thin film shielding agent is preferably used in the atomic layer deposition (ALD) process. In this case, the surface of a substrate may be effectively protected and process by-products may be effectively removed without interfering with the adsorption of a precursor compound.

The thin film shielding agent may preferably have a density of 0.8 to 2.5 g/cm³ or 0.8 to 1.5 g/cm³ and a vapor pressure (20° C.) of 0.1 to 300 mmHg or 1 to 300 mmHg.

More preferably, the thin film shielding agent may have a density of 0.75 to 2.0 g/cm³ or 0.8 to 1.3 g/cm³ and a vapor pressure (20° C.) of 1 to 260 mmHg.

In particular, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

The method of forming a thin film according to the present invention includes a step of injecting the above-described thin film shielding agent into a chamber to form a deposition layer on a substrate loaded into the chamber. In this case, the reaction speed may be improved and the thin film growth rate may be appropriately reduced through physical or chemical reactions on the substrate surface. Accordingly, even when forming a thin film under high-temperature conditions on a substrate with a complex structure, step coverage and the thickness uniformity of a thin film may be greatly improved.

In the step of shielding the thin film shielding agent on the surface of a substrate, the feeding time (sec) of the thin film shielding agent on the surface of a substrate may be preferably 0.01 to 5 seconds/cycle, more preferably 0.02 to 3 seconds/cycle, still more preferably 0.04 to 2 seconds/cycle, still more preferably 0.05 to 1 second/cycle. Within this range, thin film growth rate may be reduced, and step coverage and economics may be excellent.

In the present disclosure, the feeding time of the thin film shielding agent is based on a flow rate of 0.1 to 50 mg/cycle with a chamber volume of 15 to 20 L, more specifically, based on a flow rate of 0.8 to 20 mg/cycle with a chamber volume of 18 L.

As a preferred example, the method of forming a thin film may include step i) of vaporizing the above-described thin film shielding agent to shield the surface of a substrate loaded in a chamber; step ii) of performing 1st purging inside the chamber with a purge gas; step iii) of vaporizing a precursor compound and adsorbing the precursor compound onto an area outside the shielded area; step iv) of performing 2nd purging inside the chamber with a purge gas; step v) of supplying a reaction gas inside the chamber; and step vi) of performing 3rd purging inside the chamber with a purge gas. At this time, steps i) to vi) may be repeated as a unit cycle until a thin film of the desired thickness is obtained (see FIG. 1 below). In this way, in one cycle, when the thin film shielding agent of the present invention is injected before the precursor compound and is absorbed into the substrate, and the thin film shielding agent is sequentially injected after the precursor compound to improve film quality, even when deposition is performed at high temperatures, the thin film growth rate may be appropriately reduced, process by-products may be effectively removed, the resistivity of the thin film may be reduced, and the step coverage may be significantly improved.

As a preferred example, in the method of forming a thin film according to the present invention, in one cycle, the thin film shielding agent of the present invention may be introduced before the precursor compound and adsorbed onto the substrate. In this case, even when depositing a thin film at high temperatures, process by-products may be significantly reduced and step coverage may be significantly improved by appropriately reducing the thin film growth rate. In addition, the crystallinity of the thin film may be increased, thereby reducing the resistivity of the thin film. In addition, even when applied to semiconductor devices with a large aspect ratio, the thickness uniformity of the thin film may be greatly improved, thereby ensuring the reliability of the semiconductor device.

In the method of forming a thin film, when the thin film shielding agent is deposited before or after the deposition of the precursor compound, depending on the needs, the unit cycle may be repeated 1 to 99,999 times, preferably 10 to 10,000 times, more preferably 50 to 5,000 times, still more preferably 100 to 2,000 times. Within this range, the desired thickness of the thin film may be obtained, and the effects intended for the present invention may be sufficiently achieved.

In the present invention, for example, the chamber may be an ALD chamber, a CVD chamber, a PEALD chamber, or a PECVD chamber.

In the present invention, the thin film shielding agent or precursor compound may be vaporized, injected, and then subjected to plasma post-treatment. In this case, the growth rate of thin film may be improved and process by-products may be reduced.

When the thin film shielding agent is cross-injected onto the substrate and the precursor compound is adsorbed therebetween, the amount of purge gas injected into the chamber in the step of purging the unadsorbed thin film shielding agent is not particularly limited as long as the amount is sufficient to remove the unadsorbed thin film shielding agent. For example, the amount may be 10 to 100,000 times, preferably 50 to 50,000 times, more preferably 100 to 10,000 times. Within this range, by sufficiently removing the unadsorbed thin film shielding agent, the thin film may be formed evenly and deterioration of film quality may be prevented. Here, the input amounts of the purge gas and thin film shielding agent are each based on one cycle, and the volume of the thin film shielding agent refers to the volume of the vaporized thin film shielding agent vapor.

As a specific example, the thin film shielding agent is injected (per cycle) at a flow rate of 1.66 mL/s for an injection time of 0.5 seconds. In the step of purging the unadsorbed thin film shielding agent, when purge gas is injected (per cycle) at a flow rate 166.6 mL/s for an injection time 3 seconds, the injection amount of purge gas is 602 times the injection amount of thin film shielding agent.

In addition, in the step of purging the unadsorbed precursor compound, the amount of purge gas injected into the chamber is not particularly limited as long as the amount is sufficient to remove the unadsorbed precursor compound. For example, the amount may be 10 to 10,000 times, preferably 50 to 50,000 times, more preferably 100 to 10,000 times the volume of the precursor compound injected into the chamber. Within this range, by sufficiently removing the unadsorbed precursor compound, a thin film may be formed evenly and deterioration of film quality may be prevented. Here, the input amounts of the purge gas and precursor compound are each based on one cycle, and the volume of the precursor compound refers to the volume of the vaporized precursor compound vapor.

In addition, in the purging step performed immediately after the reaction gas supply step, the amount of purge gas introduced into the chamber may be, for example, 10 to 10,000 times, preferably 50 to 50,000 times, more preferably 100 to 10,000 times the volume of reaction gas introduced into the chamber. Within this range, the desired effects may be sufficiently achieved. Here, the input amounts of purge gas and reaction gas are based on one cycle.

The thin film shielding agent and the precursor compound may be transported into the chamber preferably by a VFC, DLI, or LDS method, more preferably an LDS method.

For example, the substrate loaded into the chamber may be heated to 50 to 400° C., as a specific example, 50 to 400° C. The thin film shielding agent or the precursor compound may be injected onto the substrate in an unheated or heated state, or may be injected unheated and then heated during the deposition process, depending on the deposition efficiency. For example, the thin film shielding agent or the precursor compound may be injected onto the substrate at 50 to 400° C. for 1 to 20 seconds.

The ratio of amount (mg/cycle) of the thin film shielding agent and the precursor compound fed into the chamber may be preferably 1:1.5 to 1:20, more preferably 1:2 to 1:15, still more preferably 1:2 to 1:12, still more preferably 1:2.5 to 1:10. Within this range, step coverage may be improved, and process by-products may be greatly reduced.

In the present invention, for example, the precursor compound may be mixed with a non-polar solvent and introduced into the chamber. In this case, the viscosity or vapor pressure of the precursor compound may be easily controlled.

The non-polar solvent may preferably include one or more selected from the group consisting of alkanes and cycloalkanes. In this case, an organic solvent with low reactivity and solubility and easy moisture management may be included. In addition, step coverage may be improved even when deposition temperature increases during thin film formation.

As a more desirable example, the non-polar solvent may include a C1 to C10 alkane or a C3 to C10 cycloalkane, preferably a C3 to C10 cycloalkane. In this case, reactivity and solubility may be low and moisture management may be easy.

In the present disclosure, C1, C3, and the like indicate the number of carbon atoms.

The cycloalkane may preferably include C3 to C10 monocycloalkanes, and among the monocycloalkanes, cyclopentane is liquid at room temperature and has the highest vapor pressure, so cyclopentane is preferable in the vapor deposition process, but the present invention is not limited thereto.

For example, the non-polar solvent may have a solubility (25° C.) of 200 mg/L or less, preferably 50 to 400 mg/L, more preferably 135 to 175 mg/L in water. Within this range, the reactivity toward the precursor compound may be reduced, and moisture may be easily managed.

In the present disclosure, solubility may be measured without any particular limitation by a measurement method or standard commonly used in the technical field to which the present invention pertains. For example, solubility may be measured by the HPLC method using a saturated solution.

Based on a total weight of the precursor compound and the non-polar solvent, the non-polar solvent may be included in an amount of 5 to 95% by weight, more preferably 10 to 90% by weight, still more preferably 40 to 90% by weight, most preferably 70 to 90% by weight.

When the content of the non-polar solvent exceeds the above range, impurities may be generated, which may increase the resistance and impurities within a thin film. When the content of the organic solvent is less than the range, the effect of improving step coverage due to solvent addition and the effect of reducing impurities such as chloride (Cl) ions may be reduced.

For example, in the method of forming a thin film, when the thin film shielding agent is used, the deposition rate reduction rate expressed by Equation 1 below may be 30% or more, as a specific example, 35% or more. In this case, by forming a deposition layer of uniform thickness as a substitution area that does not remain in the thin film due to the difference in adsorption distribution of the activator having the aforementioned structure, as a relatively coarse thin film is formed, the growth rate of a thin film formed at the same time is greatly reduced, so that even when applied to a substrate having a complex structure, the uniformity of the thin film may be secured, and the step coverage may be greatly improved. In particular, deposition in a thin thickness is possible, and the remaining amounts of O, Si, metals, and metal oxides remaining as process by-products may be improved. In addition, even the remaining amount of carbon, which was difficult to reduce in the past, may be improved.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a thin film shielding agent. DR_{f}(final deposition rate) is the deposition rate of the thin film formed by adding an oxide film thin film shielding agent during the above process. Here, the deposition rate (DR) is a value measured at room temperature and pressure using an ellipsometer for a thin film with a thickness of 3 to 30 nm, and is expressed in a unit of Å/cycle.

In the method of forming a thin film, the residual halogen intensity (c/s) in the thin film may be preferably 100,000 or less, more preferably 70,000 or less, still more preferably 50,000 or less, still more preferably 10,000 or less, as a preferred example, 5,000 or less, still more preferably 1,000 to 4,000, still more preferably 1,000 to 3,800 as measured using a thin film having a thickness of 100 Å according to SIMS. Within this range, corrosion and deterioration may be effectively prevented.

In the present disclosure, purging may be performed at preferably 1,000 to 50,000 sccm (standard cubic centimeter per minute), more preferably 2,000 to 30,000 sccm, still more preferably 2,500 to 15,000 sccm. Within this range, the thin film growth rate per cycle may be appropriately controlled. In addition, since deposition is performed as an atomic mono-layer or nearly an atomic mono-layer, the film quality may be improved.

The atomic layer deposition (ALD) process is very advantageous in manufacturing integrated circuits (ICs) that require a high aspect ratio. In particular, the ALD process has advantages such as excellent conformality, uniformity, and precise thickness control due to the self-limiting thin film growth mechanism.

For example, the method of forming a thin film may be performed at a deposition temperature of 50 to 800° C., preferably 100 to 700° C., more preferably 200 to 650° C., still more preferably 220 to 400° C., still more preferably 220 to 300° C. Within this range, a thin film with excellent film quality may be grown while implementing ALD process characteristics.

For example, the method of forming a thin film may be performed at a deposition pressure of 0.01 to 20 Torr, preferably 0.1 to 20 Torr, more preferably 0.1 to 10 Torr, most preferably 0.3 to 7 Torr. Within this range, a thin film having a uniform thickness may be obtained.

In the present disclosure, the deposition temperature and the deposition pressure may be measured as temperature and pressure formed within the deposition chamber, or as temperature and pressure applied to the substrate within the deposition chamber.

The method of forming a thin film may preferably include a step of increasing the temperature inside the chamber to the deposition temperature before introducing the thin film shielding agent into the chamber; and/or a step of purging by injecting an inert gas into the chamber before introducing the thin film shielding agent into the chamber.

In addition, as a thin film manufacturing device capable of implementing the thin film manufacturing method, the present invention may include a thin film manufacturing device including an ALD chamber, a first vaporizer for vaporizing a thin film shielding agent, a first transport means for transporting the vaporized thin film shielding agent into the ALD chamber, a second vaporizer for vaporizing a thin film precursor, and a second transport means for transporting the vaporized thin film precursor into the ALD chamber. Here, vaporizers and transport means commonly used in the technical field to which the present invention pertains may be used in the present invention without particular limitation.

When necessary, the first vaporizer for vaporizing a thin film shielding agent may be composed of at least two types: a vaporizer for vaporizing a thin film shielding agent and a vaporizer for vaporizing a thin film shielding agent.

As a specific example, the method of forming a thin film is explained. First, a substrate on which a thin film is to be formed is placed in a deposition chamber capable of atomic layer deposition.

The substrate may include a semiconductor substrate, such as a silicon substrate or a silicon oxide substrate.

The substrate may further have a conductive layer or an insulating layer formed on the upper portion thereof.

To deposit a thin film on the substrate positioned in the deposition chamber, the above-described thin film shielding agent and a precursor compound or a mixture of the precursor compound and a non-polar solvent are prepared.

Next, the prepared thin film shielding agent (for example, thin film shielding agent) is injected into the vaporizer, changed into a vapor phase, transferred to the deposition chamber, adsorbed onto the substrate, and purged to remove the unadsorbed thin film shielding agent.

Next, the prepared precursor compound or mixture (thin film-forming composition) of the precursor compound and a non-polar solvent is injected into the vaporizer, changed into a vapor phase, transferred to the deposition chamber, adsorbed onto the substrate, and shielded by a pre-injected thin film shielding agent. Then, the unadsorbed precursor compound or mixture of the precursor compound and a non-polar solvent is purged.

Next, the prepared thin film shielding agent is injected into the vaporizer, changed into a vapor phase, and delivered to the deposition chamber for adsorption, and the unadsorbed thin film shielding agent is purged.

In the present disclosure, for example, the thin film shielding agent and the precursor compound (thin film-forming composition) may be delivered to the deposition chamber by vapor flow control (VFC) using mass flow control (MFC) or a liquid delivery system (LDS) using liquid mass flow control (LMFC), preferably an LDS method.

At this time, a mixed gas of one or more selected from the group consisting of argon (Ar), nitrogen (N₂), and helium (He) may be used as a carrier gas or dilution gas to move the thin film shielding agent and the precursor compound onto the substrate, without being limited thereto.

In the present disclosure, for example, an inert gas, preferably the carrier gas or dilution gas may be used as the purge gas.

Next, a reaction gas is supplied. A reaction gas commonly used in the technical field to which the present invention pertains may be used in the present invention without particular limitation. Preferably, the reaction gas may contain a nitriding agent. The nitriding agent and the precursor compound adsorbed on the substrate react to form a nitride film.

Preferably, the nitriding agent may be nitrogen gas (N₂), hydrazine gas (N₂H₄), or a mixture of nitrogen gas and hydrogen gas.

Next, unreacted residual reaction gas is purged using an inert gas. Accordingly, in addition to excess reaction gas, generated byproducts may also be removed.

As described above, in the method of forming a thin film, for example, the step of supplying a thin film shielding agent onto a substrate, the step of purging the unadsorbed thin film shielding agent, the step of adsorbing a precursor compound/thin film-forming composition onto the substrate, the step of purging the unadsorbed precursor compound, the step of supplying a reaction gas, and the step of purging residual reaction gas may be set as a unit cycle. To form a thin film of desired thickness, the unit cycle may be repeated.

For example, the unit cycle may be repeated 1 to 99,999 times, preferably 10 to 1,000 times, more preferably 50 to 5,000 times, still more preferably 100 to 2,000 times. Within this range, the desired thin film properties may be well expressed.

In addition, the present invention provides a semiconductor substrate, and the semiconductor substrate is fabricated by the thin film formation method. In this case, the step coverage and thickness uniformity of a thin film may be excellent, and density and electrical properties may be excellent.

Preferably, the manufactured thin film may a thickness of 20 nm or less, a resistivity value of 50 to 400 μΩ·cm based on a thin film thickness of 10 nm, a halogen content of 10,000 ppm or less, and a step coverage of 90% or more. Within this range, the thin film has excellent performance as a diffusion barrier and has the effect of reducing corrosion of metal wiring materials, without being limited thereto.

For example, the thin film may have a thickness of 0.1 to 20 nm, preferably 1 to 20 nm, more preferably 3 to 25 nm, still more preferably 5 to 20 nm. Within this range, thin film properties may be excellent.

For example, based on a thin film thickness of 10 nm, the thin film may have a resistivity value of 0.1 to 400 μΩ·cm, preferably 15 to 300 μΩ·cm, more preferably 20 to 290 μΩ·cm, still more preferably 25 to 280 μΩ·cm. Within this range, thin film properties may be excellent.

The thin film may have a halogen content of preferably 10,000 ppm or less or 1 to 9,000 ppm, still more preferably 5 to 8,500 ppm, still more preferably 100 to 1,000 ppm. Within this range, thin film properties may be excellent, and thin film growth rate may be reduced. Here, the halogen remaining in the thin film may be, for example, Cl₂, Cl, or Cl—. As the halogen residue within the thin film decreases, the film quality increases.

For example, the thin film may have a step coverage of 90% or more, preferably 92% or more, more preferably 95% or more. Within this range, since even a thin film of complex structure may be easily deposited on a substrate, the thin film may be applied to next-generation semiconductor devices.

Preferably, the manufactured thin film may have a thickness of 20 nm or less, a carbon, nitrogen, and halogen content of 10,000 ppm or less based on a thin film thickness of 10 nm, and a step coverage of 90% or more. Within this range, the thin film may have excellent performance as a dielectric film or blocking film, without being limited thereto.

For example, when necessary, the thin film may have a multilayer structure of two or more layers. As a specific example, the multilayer film with a two-layer structure may have a lower layer-middle layer structure, and the multilayer film with a three-layer structure may have a lower layer-middle layer-upper layer structure.

For example, the lower layer may be formed of one or more selected from the group consisting of Si, SiO₂, MgO, Al₂O₃, CaO, ZrSiO₄, ZrO₂, HfSiO₄, Y₂O₃, HfO₂, LaLuO₂, Si₃N₄, SrO, La₂O₃, Ta₂O₅, BaO, and TiO₂.

For example, the middle layer may be formed of TiₓN_{y}, preferably TN.

For example, the upper layer may be formed of one or more selected from the group consisting of W and Mo.

Hereinafter, preferred examples and drawings are presented to help understand the present invention, but the following examples and drawings are only illustrative of the present invention, and it is obvious to those skilled in the art that various changes and modifications are possible within the scope and technical idea of the present invention. Such changes and modifications fall within the scope of the appended patent claims.

### EXAMPLES

### Examples 1 and 2, Comparative Examples 1 to 3

An ALD deposition process was performed according to FIG. 1 below using the components shown in Table 1 below.

FIG. 1 below is a schematic diagram showing a deposition process sequence according to the present invention, focusing on one cycle.

Specifically, as the thin film shielding agent, a compound represented by Chemical Formula 1-4 below and a compound represented by Chemical Formula 3-1 below were prepared.

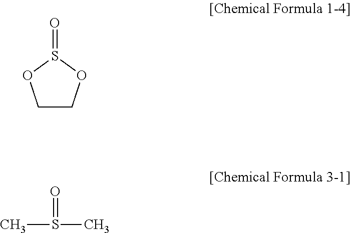

In addition, as the precursor, tris(dimethylamino)cyclopentadienyl hafnium (CpHf(NMe₂)₃, indicated as CpHf in the table below) and trimethyl aluminum (indicated as TMA in the table below) were prepared.

Argon was introduced into the chamber at a rate of 5000 ml/min, and a vacuum pump was used to create a thin, inert atmosphere by maintaining the pressure inside the chamber at 1.5 Torr.

The thin film shielding agent shown in Table 1 below was placed in a canister and the partial pressure and temperature were adjusted to achieve the injection amount (mg/cycle). Then, the thin film shielding agent was applied into a substate loaded into the chamber for 1 second, and the chamber was purged for 10 seconds.

Next, the precursor compound was placed in the canister and injected into the deposition chamber through a vapor flow controller (VFC) as shown in Table 1, and the chamber was purged for 10 seconds.

Next, the concentration of O₃ in O₂ as a reactive gas was adjusted to 200 g/m³ and the reactive gas was introduced into the deposition chamber as shown in Table 1, and the chamber was purged for 10 seconds. At this time, the substrate on which a thin film was to be formed was heated under the temperature conditions shown in Table 1 below.

This process was repeated 100 to 400 times to form a self-limiting atomic layer thin film with a thickness of 10 nm.

For each thin film of Examples 1 and 2 and Comparative Examples 1 to 3, the deposition rate reduction rate (D/R reduction rate), SIMS C impurities, and step coverage were measured in the following methods, and the results are shown in Table 1.

Deposition rate reduction rate (D/R (dep. rate) reduction rate): The deposition rate reduction rate represents the rate of reduction in the deposition rate after the shielding agent is added compared to the deposition rate before the shielding agent is added, and was calculated as a percentage using the measured Å/cycle values.

Specifically, using an ellipsometer which is a device capable of measuring optical properties such as thickness and refractive index of the thin film by using the polarization characteristics of light for the manufactured thin film, the thickness of the thin film was measured. Then, the thickness of the thin film deposited per cycle was calculated by dividing the measured thickness by the number of cycles. Based on these results, the thin film growth rate reduction rate was calculated. Specifically, the calculation was performed using Equation 1 below.

$$Deposition rate reduction rate = [ \{ ( {DR}_{i} ) - ( D {R}_{f} ) \} / ( {DR}_{i} ) ] \times 100 \qquad [ Equation 1 ]$$

In Equation 1, deposition rate (DR, Å/cycle) is the speed at which a thin film is deposited. In the deposition of a thin film formed from a precursor and a reactant, DRᵢ (initial deposition rate) is the deposition rate of the thin film formed without adding a thin film shielding agent. DR_{f}(final deposition rate) is the deposition rate of the thin film formed by adding an oxide film thin film shielding agent during the above process. Here, the deposition rate (DR) is a value measured at room temperature and pressure using an ellipsometer for a thin film with a thickness of 3 to 30 nm, and is expressed in a unit of Å/cycle.

* To evaluate the non-uniformity, the maximum and minimum thicknesses were selected from the thicknesses of the thin film measured by the ellipsometer, and the results calculated using Equation 2 below are shown in Table 1 below. Specifically, the thickness of four locations on the east, west, south, and north edges of a 300 mm wafer and the thickness of one location in the center were measured.

$$Non ‐ uniformity ( \% ) = [ \{ ( Maximum thickness - Minimum thickness ) / 2 \} \times Average thickness ] \times 100 \qquad [ Equation 2 ]$$

*SIMS (Secondary-ion mass spectrometry) C impurities: The C impurity value was checked from the SIMS graph by considering the C impurity content (counts) when the thin film was axially penetrated by an ion sputter and the sputter time was 50 seconds with little contamination on the substrate surface layer, and is shown in Table 1 below.

* Step coverage (%): TEM measurements were taken on specimens cut horizontally at positions 100 nm downward from the upper portion (left drawing) and 100 nm upward from the lower portion (right drawing) of thin films deposited on a substrate of a complex structure with an aspect ratio of 22:1 by Examples 1 and 2 and Comparative Examples 1 to 3, and the step coverage was measured by Equation 3 below.

$$Step coverage \% = ( Thickness deposited on inner wall of lower portion / Thickness deposited on inner wall of upper portion ) \times 100 \qquad [ Equation 3 ]$$

TABLE 1

| | | | | | | Re- | Shield- | | | | |
|---|---|---|---|---|---|---|---|---|---|---|---|
| | | | | | Pre- | action | ing | | | | |
| | | | | | cursor | gas | agent | Depo- | | | Step |
| | | | | Depo- | injec- | injec- | injec- | sition | Non- | Equation | cov- |
| | | Re- | Shield- | sition | tion | tion | tion | rate | uni- | 1 | erage |
| | Pre- | action | ing | temper- | con- | con- | con- | (Å/ | formity | calcu- | (S/C, |
| No. | cursor | gas | agent | ature | dition | dition | dition | cycle) | (%) | lation | %) |
| Com- | CpHf | O₃ | — | 420° C. | VFC, | 5 s, | — | 0.93 | 1.22 | — | 23.6 |
| parative | | | | | 3 s, | 500 | | | | | |
| Exam- | | | | | 100 | sccm | | | | | |
| ple1 | | | | | sccm | | | | | | |
| Com- | CpHf | O₃ | Chem- | 420° C. | VFC, | 5 s, | 15 mg/ | 0.745 | 8.33 | 20% | 82.2 |
| parative | | | ical | | 3 s, | 500 | cycle | | | | |
| Exam- | | | For- | | 100 | sccm | | | | | |
| ple2 | | | mula | | sccm | | | | | | |
| | | | 3-1 | | | | | | | | |
| Exam- | CpHf | O₃ | Chem- | 420° C. | VFC, | 5 s, | 15 mg/ | 0.51 | 1.93 | 45% | 98.4 |
| ple1 | | | ical | | 3 s, | 500 | cycle | | | | |
| | | | For- | | 100 | sccm | | | | | |
| | | | mula | | sccm | | | | | | |
| | | | 1-4 | | | | | | | | |
| Com- | TMA | O₃ | — | 440° C. | VFC, | 5 s, | — | 0.75 | 1.20 | — | 95.0 |
| parative | | | | | 2 s, | 300 | | | | | |
| Exam- | | | | | 100 | sccm | | | | | |
| ple3 | | | | | sccm | | | | | | |
| Exam- | TMA | O₃ | Chem- | 440° C. | VFC, | 5 s, | 15 mg/ | 0.49 | 2.52 | 35% | 100.5 |
| ple2 | | | ical | | 2 s, | 300 | cycle | | | | |
| | | | For- | | 100 | sccm | | | | | |
| | | | mula | | sccm | | | | | | |
| | | | 1-4 | | | | | | | | |

In the table, CpHf is an abbreviation for tris(dimethylamido)cyclopentadienyl hafnium and TMA is an abbreviation for trimethyl aluminum. As shown in Table 1, compared to Comparative Examples 1 and 3 in which the thin film shielding agent of the present invention is not used, Examples 1 and 2 using the thin film shielding agent of the present invention exhibited improved deposition rate reduction rate and excellent impurity reduction characteristics.

Specifically, the non-uniformity of Example 1 is 1.93%, which is very low compared to the non-uniformity of Comparative Example 1, which is 8.33%. In addition, in Example 1, the GPC reduction effect is 45%, showing improvement compared to Comparative Example 1 (20%)

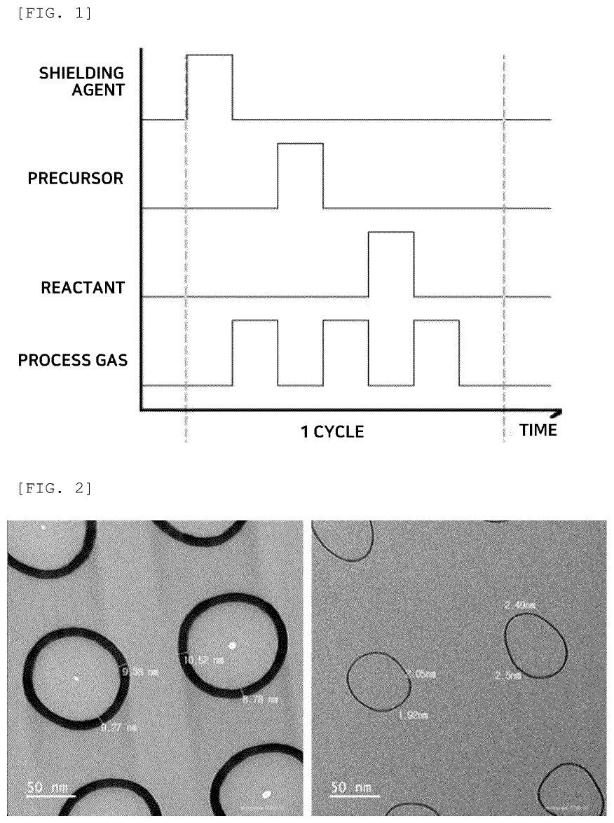

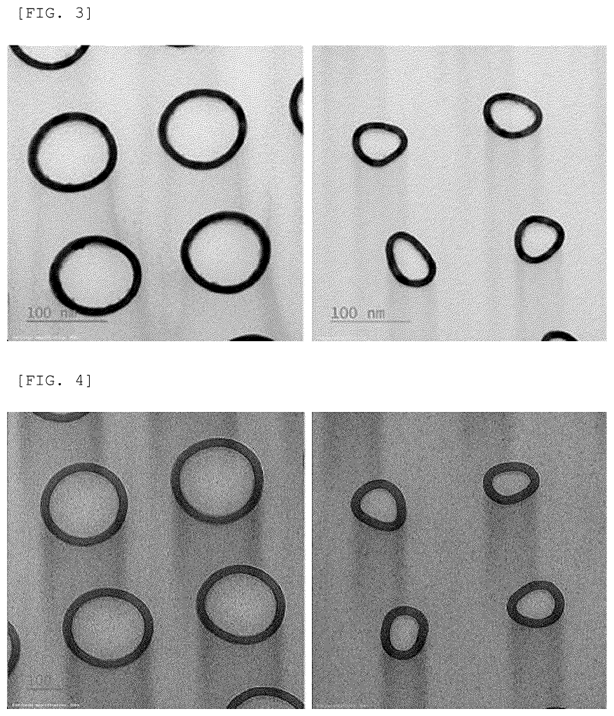

Specifically, as shown in FIGS. 2 to 4, compared to Comparative Example 1 not using the thin film shielding agent, in the thin films formed in Examples 1 and 2 using the thin film shielding agent according to the present invention, both the upper and lower thicknesses increased.

In addition, compared to Comparative Example 2, in which an incompatible substance such as dimethyl sulfoxide was used, Examples 1 and 2 using the thin film shielding agent according to the present invention exhibited improved deposition rate reduction rate and excellent impurity reduction characteristics.

Specifically, the step coverage (45%) of Example 1 using the thin film shielding agent according to the present invention was increased by more than twice that (20%) of Comparative Example 2 using dimethyl sulfoxide.

1. A thin film shielding agent for depositing a metal oxide film or non-metal oxide film on a substrate,
wherein the thin film shielding agent comprises one or more elements having electronegativity between electronegativity of a metal or non-metal constituting the oxide film and electronegativity of oxygen and shields the deposition.

2. The thin film shielding agent according to claim 1, wherein the metal or non-metal shields a surface of a thin film formed using one or more precursor compounds selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

3. The thin film shielding agent according to claim 1, wherein the thin film shielding agent is a compound comprising four or more elements having an electronegativity of 2.1 to 3.1.

4. The thin film shielding agent according to claim 1, wherein the thin film shielding agent is a compound having a structure represented by Chemical Formula 1 below. 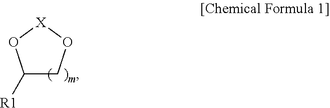
wherein R1 is H, OH, CH₃, OCH₃, OCH₂CH₃, OCH₂CH₂CH₃, an alkyl group having 1 to 5 carbon atoms, an alkene group having 1 to 5 carbon atoms, or an alkane group having 1 to 5 carbon atoms; X is 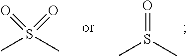
and m is an integer from 0 to 4.

5. The thin film shielding agent according to claim 1, wherein the thin film shielding agent comprises one or more selected from compounds represented by Chemical Formulas 1-1 to 1-9 below.

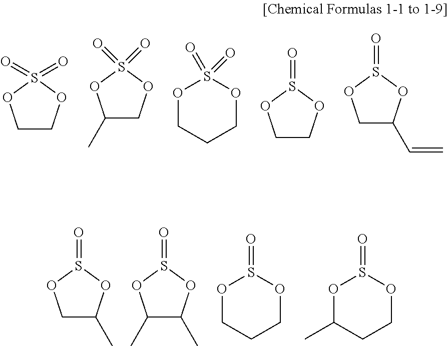

6. A thin film-forming composition, comprising:
a precursor compound constituting a thin film deposition layer; and a thin film shielding agent,
wherein the thin film shielding agent is the thin film shielding agent according to claim 1; and
the precursor compound is a compound represented by Chemical Formula 2 below. 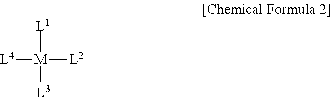
wherein M comprises one or more selected from Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd; and L1, L2, L3, and L4 are —H, —X, —R, —OR, —NR, or Cp (cyclopentadiene) and are the same or different,
wherein —X is F, Cl, Br, or I; —R is C1-C10 alkyl, C1-C10 alkene, or C1-C10 alkane and is linear or cyclic; and L1, L2, L3, and L4 are formed from 2 to 6 depending on an oxidation number of a central metal (M).

7. The thin film-forming composition according to claim 6, wherein the thin film shielding agent provides a shielded area for an oxide film, a nitride film, a metal film, or a selective thin film thereof, and the shielded area is formed on an entire or part of a substrate on which the oxide film, the nitride film, the metal film, or the selective thin film thereof is formed.

8. The thin film-forming composition according to claim 6, wherein, based on 100% of a total area of the substrate, the shielded area occupies 10 to 95% of the entire substrate or a portion of the substrate, and an unshielded area occupies a remainder.

9. The thin film-forming composition according to claim 6, wherein the thin film improves step coverage in a process of forming a laminated film of one or more selected from the group consisting of Al, Si, Ti, V, Co, Ni, Cu, Zn, Ga, Ge, Se, Zr, Nb, Mo, Ru, Rh, In, Sn, Sb, Te, Hf, Ta, W, Re, Os, Ir, La, Ce, and Nd.

10. A method of forming a thin film, comprising injecting one or more thin film shielding agents selected from compounds having a structure represented by Chemical Formula 1 below and a precursor compound into a chamber to form a deposition layer on a substrate loaded into the chamber. 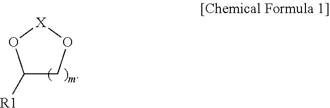
wherein R1 is H, OH, CH₃, OCH₃, OCH₂CH₃, OCH₂CH₂CH₃, an alkyl group having 1 to 5 carbon atoms, an alkene group having 1 to 5 carbon atoms, or an alkane group having 1 to 5 carbon atoms; X is 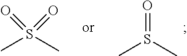
and m is an integer from 0 to 4.

11. The method according to claim 10, wherein the precursor compound and the thin film shielding agent are independently transported into the chamber by a VFC, DLI, or LDS method, and the thin film is a silicon nitride film, a silicon oxide film, a titanium nitride film, a titanium oxide film, a tungsten nitride film, a molybdenum nitride film, a hafnium oxide film, a zirconium oxide film, a tungsten oxide film, or an aluminum oxide film.

12. A semiconductor substrate, comprising the thin film formed by the method according to claim 10.

13. The semiconductor substrate according to claim 12, wherein the thin film has a multilayer structure of two or more layers.

14. A semiconductor device, comprising the semiconductor substrate according to claim 12.
